(19) United States
(12) Plant Patent Application Publication
(10) Pub. No.: US 2002/0148015 P1
(43) Pub. Date: Oct. 10, 2002

(54) COMPACT FLORIBUNDA ROSE PLANT NAMED 'POULAC001'

(75) Inventors: L. Pernille Olesen, Fredensborg (DK); Mogens N. Olesen, Fredensborg (DK)

Correspondence Address: Mogens N. Olesen, Poulsen Roser Pacific, Inc., 2550 Old Military Road, Central Point, OR, 97502, US

(73) Assignee: Poulsen Roser ApS.

(21) Appl. No.: 09/776,157

(22) Filed: Feb. 2, 2001

(51) Int. Cl. A01H 5/00

(52) U.S. Cl. PLT/148

(57) ABSTRACT

A new compact floribunda rose which has abundant, light pink flowers and attractive foliage. This new and distinct variety has shown to be uniform and stable in the resulting generations from asexual propagation.

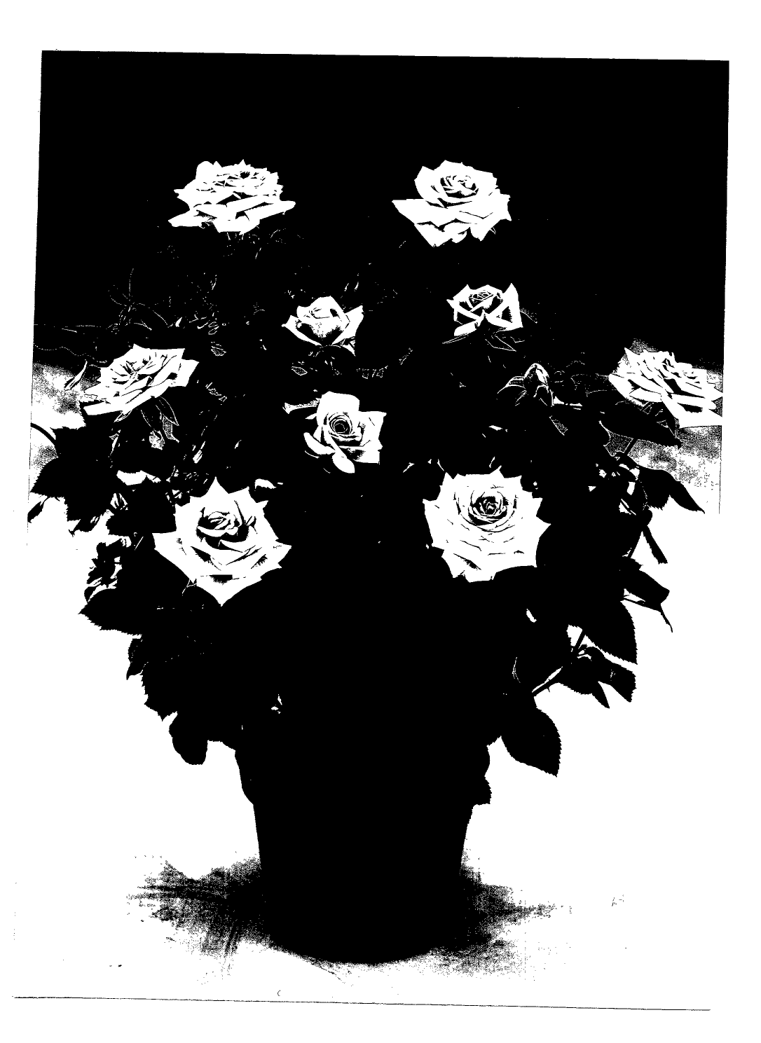

### SUMMARY OF THE INVENTION

The present invention constitutes a new and distinct variety of floribunda rose plant which originated from a controlled crossing between ‘POULfiry’ described and illustrated in Plant patent application Ser. 09/277,237, dated Mar. 26, 1999 and ‘POULsint’ an unpatented variety. The two parents were crossed during the summer of 1996 and the resulting seeds were planted in a controlled environment in Fredensborg, Denmark. The new variety is named ‘POULacOOl’.

The new rose may be distinguished from its seed parent, ‘POULfiry’, by the following combination of characteristics:

1. ‘POULac001’ has pink flowers, while ‘POULfiry’ has bright orange flowers.

2. ‘POULacOOl’, as a compact floribunda is taller in habit when compared to the miniature rose ‘POULfiry’.

3. ‘POULfiry’ exhibits more anthocyanin in the foliage when compared to ‘POULac001’.

The new variety may be distinguished from its pollen parent, ‘POULsint’ by the following combination of characteristics:

1. ‘POULac001’ exhibits light pink flowers, whereas the pollen parent exhibits orange-red flowers.

2. ‘POULac001’ has a larger flower diameter than that of ‘POULsint’.

3. ‘POULac001’ has glossy foliage, whereas ‘POULsint’ displays matte foliage.

The objective of the hybridization of this rose variety was to create a new and distinct variety for garden use with unique qualities, such as:

1. Uniform and abundant pink flowers;

2. Vigorous, but compact growth when propagated both as a budded rose and on its own roots;

3. Disease resistance. 4. Suitability for production in pots.

This combination of qualities is not present in previously available commercial cultivars of this type and distinguish ‘POULac001’ from all other varieties of which we are aware.

As part of their rose development program, L. Pernille Olesen and Mogens N. Olesen germinated the seeds from the aforementioned hybridization during winter 1996 and conducted evaluations on the resulting seedlings in a controlled environment in Fredensborg, Denmark.

‘POULac001’ was selected in the spring 1997 by the inventors as a single plant from the progeny of the aforementioned hybridization.

Asexual reproduction of ‘POULac001’ by traditional budding and rooted cuttings was first done by L. Pernille and Mogens N. Olesen in their nursery in Fredensborg, Denmark in July 1997. This initial and other subsequent asexual propagations conducted in controlled environments have demonstrated that the characteristics of ‘POULac001’ are true to type and are transmitted from one generation to the next.

### BRIEF DESCRIPTION OF THE DRAWING

The accompanying color illustration shows as true as is reasonably possible to obtain in color photographs of this type the variety rose ‘POULac001’. Specifically illustrated in SHEET 1 include:

1. Rose plant exhibiting foliage, leaves, and leaflets.

| | | Very double |
|---|---|---|
| Petalage | Double, 25+ | 50-60 petals |
| Flower size | 8-10 cm | 6-8 cm |

Parents:

Seed parent.—‘POULfiry’.

Pollen parent.—‘POULsint’.

Classification:

Botanical.—Rosa hybrida.

Commercial.—Compact floribunda.

### FLOWER AND FLOWER BUD

Blooming habit: Continuous.

Flower bud:

Bud form.—Ovate.

Sepals.—Absent to very weak.

Receptacle.—Size: Small.

Peduncle.—Surface: Average to above average abundance of hairs and prickles.

Borne.—Few flowering shoots.

2. Flower bud, partially opened bud, and open bloom;

3. General plant growth and habit.

### DETAILED DESCRIPTION OF THE VARIETY

The following is a description of ‘POULac001’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULskov’, a rose variety from the same inventors described and illustrated in U.S. Plant Pat. No. 9,062 and issued on Feb. 28, 1995 are compared to ‘POULac001’ in Chart 1.

CHART 1

| | ‘POULac001’ | ‘POULskov’ |
|---|---|---|
| Color of open | Red Group 55B | Red Group 56B-56D |
| flower, outer | | |
| side, middle | | |
| zone. | | |

Flower bloom:

Fragrance.—Absent to very weak.

Size.—Medium. Average flower diameter is between 8-10 cm when open.

Form.—Shape of flower when viewed from the side: Upper part: Convex. Lower part: Flat. Viewed from above: Star-shaped. Petalage: Double. Average range: 25+ petals under normal conditions.

Color:

Petals.—Inner Side: Middle zone: Red Group 54D. Marginal zone: Red Group 54D. Basal petal spot: Yellow Group 6B. Outer side: Middle zone: Red Group 55B. Marginal zone: Red Group 55B. Basal petal spot: Yellow Group 4C.

General Tonality: On open flower between Red Group 54D and 55B.

Petals:

Petal reflex.—Strong.

Undulation of margin.—Weak.

Reproductive Organs:

Filaments.—Color: Yellow

Hips.—Shape: Pitcher-shaped.

### PLANT

Plant growth: Dense, compact, and even growth. When grown as 15-cm pot plant the average height of the plant is 40 cm and the average width is 60 cm.

Stems:

Thorns.—Incidence: Few. Size: Small and medium. Shape: Concave.

Plant foliage:

Leaf size.—Medium.

Abundance.—Average.

Glossy.—Upper side weak glossiness.

Color.—Upper Leaf Surface: Medium to dark green. Juvenile foliage: Exhibits anthocyanin on young shoots. Anthocyanin intonation: Color: Bronze to reddish brown.

Plant Leaflets

Leaflet.—Cross Section: Flat. Margin Undulation: Moderate.

Terminal leaflet.—Length of blade: Medium. Width of blade: Medium. Shape: Rounded.

Disease resistance: Above average resistance to mildew, rust, black spot, and Botrytis under normal growing conditions in Fredensborg, Denmark and Hannover, Germany.

Cold Hardiness: The variety ‘POULac001’ has been found to be cold hardy in Fredensborg, Denmark and Hannover, Germany.

1. A new and distinct variety of rose plant of the compact floribunda rose class, substantially as herein illustrated and described as a distinct and novel rose variety due to its abundant light pink flowers, disease resistance, and extended period of bloom.
